(19) United States
(12) Patent Application Publication
(10) Pub. No.: US 2026/0296087 A1
(43) Pub. Date: Oct. 1, 2026

(54) SYSTEMS AND METHODS FOR COLLECTING AND STORING BIOMETRIC IDENTIFIERS

(71) Applicant: Stillwater Solutions 23 LLC d/b/a National Child ID Program, Waco, TX (US)

(72) Inventors: Kenny HANSMIRE, Waco, TX (US); Mark SALMANS, Waco, TX (US)

(73) Assignee: Stillwater Solutions 23 LLC d/b/a National Child ID Program, Waco, TX (US)

(21) Appl. No.: 19/535,842

(22) Filed: Feb. 10, 2026

Related U.S. Application Data
(60) Provisional application No. 63/757,247, filed on Feb. 11, 2025.

(51) Int. Cl. B42D 25/313; A61B 5/1172; B42D 15/04; C12Q 1/6806

(52) U.S. Cl. CPC B42D 25/313; A61B 5/1172; B42D 15/045; C12Q 1/6806

(57) ABSTRACT

Systems and methods for collecting one or more biometric identifiers of a user are provided. A system may include an apparatus that includes a first area for the collection of a first biometric identifier and a second area for the collection of a second biometric identifier of the user. The apparatus may include a compound applied as a layer to at least a first portion of the first area. In a first configuration of the apparatus, the second area is presented for the collection of the second biometric identifier of the user, and in a second configuration of the collection apparatus, the second area is not in contact with the first area. The system may include an applicator that, when in contact with a portion of the layer of the compound, interacts with the portion of the layer such that an impression of the first biometric identifier of the user is captured.

### CROSS-REFERENCE TO RELATED APPLICATIONS

This application claims the benefit of and priority to U.S. Provisional Application No. 63/757,247, filed Feb. 11, 2025, which is incorporated by reference herein in its entirety for all purposes.

### BACKGROUND

Personally identifiable biometric identifiers such as fingerprints and Deoxyribonucleic acid (DNA) can be utilized for a variety of beneficial uses. For example, in the event of a missing child, this information provides law enforcement and families with unique, reliable identifiers to aid in locating and quickly identifying the missing child. Additionally, these personally identifiable biometric identifiers have proven useful in reuniting families during emergencies or natural disasters where family members, such as children, may become separated from their caregivers. However, the effective use of personally identifiable biometric identifiers is often predicated on having access to a pre-collected sample of one or more personally identifiable biometric identifiers that can be used to compare against an unknown biometric identifier.

Despite the known benefits of using personally identifiable biometric identifiers, widespread collection and safe, long-term storage of personally identifiable biometric identifiers of persons, particularly children, has proven to be difficult in traditional collection and storage systems. Guardians of minors are often reluctant to have an organization or agency collect and store biometric data of minors because of inconvenience or privacy concerns associated with centralized collection and storage. Beyond the privacy concerns that may be associated with the centralized collection and storage of personally identifiable biometric identifiers, centralized efforts are labor and monetarily intensive efforts that have traditionally impeded such efforts. Even when a biometric identifier is privately collected, long-term storage proves to be difficult because of the possibility of contamination, particularly when multiple different identifiers are collected.

### SUMMARY

For at least the reasons above, there is a need for a system and method for facilitating widespread, private collection and safe, long-term storage of biometric identifiers. For example, in at least one implementation of the systems and methods described herein, a collection document for collecting biometric identifiers includes at least two distinct areas for collection of two distinct biometric identifiers, such as, by way of a non-limiting example, a fingerprint and a saliva sample. The two areas may be positioned upon the collection document such that the two areas are not in contact with one another when in a collection configuration and/or storage configuration, thus reducing the possibility of cross-contamination of the two samples. In at least one non-limiting implementation of the systems and methods described herein, the collection document may be a pre-folded pamphlet (e.g., a tri-fold pamphlet) with a first area for collecting one or more fingerprints of a user with an inkless fingerprint collection compound. The pre-folded collection document may also have a second area for collecting a saliva sample of the user. The second area may be positioned on a corner of the pre-folded collection document such that the second area may be placed in a mouth of a user, thereby facilitating a deposit of a saliva sample into/onto the second area. To avoid contamination of the saliva sample with the fingerprint collection compound (or vice versa), the second area may be positioned on an upper corner of the document and the first section may be positioned on a lower portion of the document. In this manner, the two collection areas are physically separated from each other in the collection configuration (e.g., in the unfolded position) and/or the storage configuration (e.g., in the folded position), thus reducing the likelihood of cross-contamination.

In some aspects, the techniques described herein relate to a system including: a collection apparatus including: a first area for a first collection of a first biometric identifier of a user, the first area positioned at a first location of the collection apparatus; a compound applied as a layer to at least a first portion of the first area; a second area for a second collection of a second biometric identifier that includes DNA of the user; wherein the second area is positioned at a second location of the collection apparatus, the second location of the collection apparatus positioned such that in a first configuration of the collection apparatus, the second area is presented for the second collection of the second biometric identifier that includes the DNA of the user; wherein the second location of the collection apparatus is positioned such that in a second configuration of the collection apparatus, the second area is not in contact with the first area; and an applicator that, when in contact with a portion of the layer of the compound, interacts with the portion of the layer of the compound such that an impression of the first biometric identifier of the user is captured.

In some aspects, the techniques described herein relate to a system, wherein the collection apparatus further includes a third area for collection of a third biometric identifier of a second user.

In some aspects, the techniques described herein relate to a system, wherein the collection apparatus further includes a fourth area for collection of a fourth biometric identifier that includes DNA of the second user.

In some aspects, the techniques described herein relate to a system, wherein the second user is a guardian or parent of the user.

In some aspects, the techniques described herein relate to a system, wherein the third area is positioned on the collection apparatus at a third location of the collection apparatus; wherein the third location of the collection apparatus is positioned such that in the second configuration at least a portion of the third area is not in contact with the first area.

In some aspects, the techniques described herein relate to a system, further including a covering configured to be applied to at least one or more of the first area or the second area such that the covering prevents direct contamination of at least the second biometric identifier with another biometric identifier.

In some aspects, the techniques described herein relate to a system, wherein the covering is an expandable enclosure that is coupled to the collection apparatus and configured to receive at least a second portion of the second area.

In some aspects, the techniques described herein relate to a system, wherein the covering is a flap coupled to and extending from the collection apparatus such that in a first position the flap exposes the second area for collection of the second biometric identifier and in a second position the flap covers the second area.

In some aspects, the techniques described herein relate to a system, wherein the covering is at least partially removeably coupled to the collection apparatus such that when the covering is coupled to the collection apparatus it covers at least one of a portion of the first area or the second area.

In some aspects, the techniques described herein relate to a system, wherein the second area is partitioned into a plurality of segments.

In some aspects, the techniques described herein relate to a system, wherein the plurality of segments are demarcated by one or more demarcation elements.

In some aspects, the techniques described herein relate to a system, wherein the one or more demarcation elements include at least one of a perforation, a line, and a slit.

In some aspects, the techniques described herein relate to a system, wherein the one or more demarcation elements include a geometric shape for removal from the second area by a punch.

In some aspects, the techniques described herein relate to a system, wherein the collection apparatus includes an expandable enclosure for collection of a fifth biometric identifier of the user.

In some aspects, the techniques described herein relate to a system, wherein the fifth biometric identifier is a biological sample, including at least one of a tooth sample, a hair sample, a skin sample, a blood sample, or a saliva sample.

In some aspects, the techniques described herein relate to a system, wherein the collection apparatus is pre-folded at one or more locations.

In some aspects, the techniques described herein relate to a system, wherein the collection apparatus is pre-folded at two locations such that the collection apparatus is divided into three panels.

In some aspects, the techniques described herein relate to a system, wherein the first area is positioned on at least a third portion of a first panel and a second panel, and the second area is positioned on at least a fifth portion of a third panel.

In some aspects, the techniques described herein relate to a system, wherein at least a portion of the second area has provided thereon a flavoring agent.

In some aspects, the techniques described herein relate to a system, wherein the applicator is an alcohol-based inkless activation solution.

In some aspects, the techniques described herein relate to a system, wherein the collection apparatus includes an identifier for an electronic page for inputting data associated with the collection apparatus.

In some aspects, the techniques described herein relate to a system, wherein the electronic page displays an interactive graphical component that, when selected, stores the inputted data to a storage database.

In some aspects, the techniques described herein relate to a system, further including a storage device configured to store the collection apparatus, wherein at least a portion of the storage device includes a protective lining configured to protect the second area of the collection apparatus from one or more environmental factors.

In some aspects, the techniques described herein relate to a method for manufacturing a system for collecting and storing one or more biometric identifiers, the method including: forming a collection document having a portion configured to be positioned in a mouth of a first user, the collection document having a first panel, a second panel, and a third panel, wherein the portion is located at least in part on the third panel, and wherein the portion is configured to collect a first biometric identifier of the first user while the collection document is positioned within the mouth of the first user; wherein the collection document is formed such that the collection document may be adjusted from an open configuration into a closed configuration by adjusting the collection document including: folding the third panel such that a first face of the third panel is positionally opposing a first face of the second panel; and folding the first panel such that a first face of the first panel is positionally opposing a second face of the third panel, wherein the first biometric identifier that is deposited on the third panel is positionally opposing a first section of the first face of the second panel that does not include a biometric identifier such that the third panel is positioned between the first panel and the second panel while in the closed configuration and such that the portion of the third panel with the first biometric identifier of the first user is at least partially protected from one or more environmental factors.

In some aspects, the techniques described herein relate to a method, wherein the one or more environmental factors include one or more of light, moisture, heat, humidity, contaminants, and dust.

In some aspects, the techniques described herein relate to a method, wherein the collection document is configured to be adjusted into an open configuration, such that the portion of the third panel is positioned outside of the first panel and the second panel.

In some aspects, the techniques described herein relate to a method, wherein the collection document includes a second portion configured to collect a second biometric identifier, wherein the second portion is positioned at least partially upon the second panel such that the second biometric identifier is not in contact with the first biometric identifier when the collection document is in the closed configuration.

In some aspects, the techniques described herein relate to a method, wherein the collection document includes a second portion where a second biometric identifier may be collected, and wherein the second portion is positioned on the third panel such that the third panel is positioned between the first panel and the second panel while in the closed configuration such that the second portion of the third panel is at least partially protected.

### BRIEF DESCRIPTION OF THE FIGURES

FIG. 1 illustrates a system for collecting and storing one or more biometric identifiers, according to an embodiment.

FIG. 2A illustrates a first side of a collection document for collecting one or more biometric identifiers, according to an embodiment.

FIG. 2B illustrates a second side of a collection document for collecting one or more biometric identifiers, according to an embodiment.

FIG. 3A illustrates a collection document with a segmented collection area for collecting a biometric identifier, according to an embodiment.

FIG. 3B illustrates a collection document with a segmented collection area for collecting a biometric identifier, according to an embodiment.

FIG. 4A illustrates a protective covering to prevent cross-contamination between multiple biometric identifiers, according to an embodiment.

FIG. 4B illustrates a protective covering to prevent cross-contamination between multiple biometric identifiers, according to an embodiment.

FIG. 4C illustrates a protective covering to prevent cross-contamination between multiple biometric identifiers, according to an embodiment.

FIG. 4D illustrates a collection document for collecting one or more biometric identifiers, according to an embodiment.

FIG. 5 illustrates a flow diagram of a method for collecting and storing one or more biometric identifiers, according to an embodiment.

FIG. 6A illustrates a first side of a collection document for collecting one or more biometric identifiers, according to an embodiment.

FIG. 6B illustrates a second side of a collection document for collecting one or more biometric identifiers, according to an embodiment.

FIG. 7 illustrates a flow diagram of a method for manufacturing a collection document for collecting and storing one or more biometric identifiers, according to an embodiment.

### DETAILED DESCRIPTION

Before turning to the figures, which illustrate certain exemplary embodiments in detail, it should be understood that the present disclosure is not limited to the details or methodology set forth in the description or illustrated in the figures. It should also be understood that the terminology used herein is for the purpose of description only and should not be regarded as limiting.

Referring generally to the figures, a system and method for facilitating widespread, private collection and save, long-term storage of personally identifiable biometric identifiers (referred to herein generally as “biometric identifiers”) are shown. It shown be understood that the embodiments described herein and shown in the figures are exemplary and provided for illustrative purposes. Any references to particular embodiments should not be construed as limiting the features or aspects described herein and shown in the figures to any particular embodiment. Features or aspects of various embodiments herein may be omitted from particular embodiments or combined with features or aspects discussed in connection with other embodiments, and such omissions or combinations are within the scope of the present disclosure.

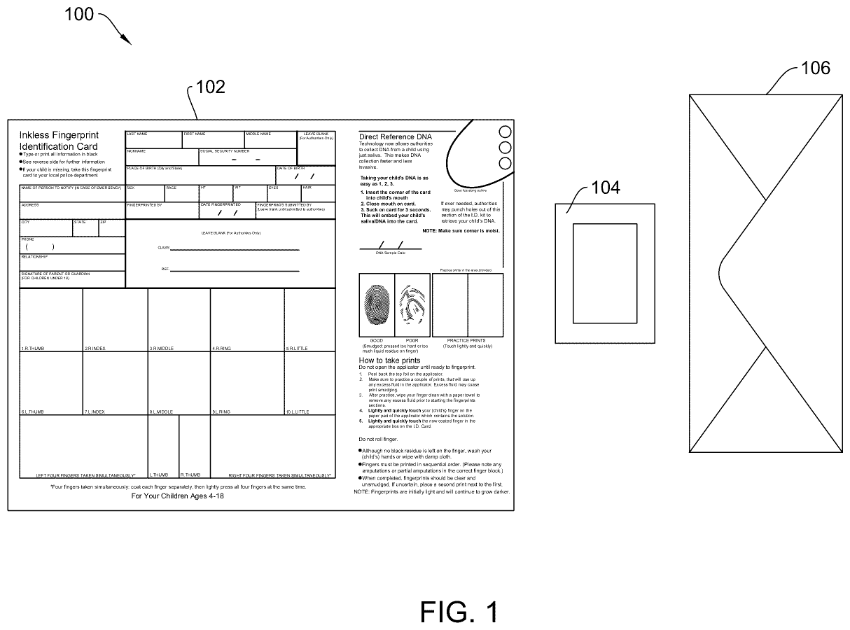

Turning to FIG. 1, a system 100 for collecting and safely storing one or more biometric identifiers of one or more individuals is shown. The system 100 may include, for example, an apparatus for collecting biometric identifiers (shown as a collection apparatus 102), a collection activator (shown as an applicator 104) and/or a device for storing the collected biometric identifiers (shown as a storage device 106).

The various components of the system 100 may be used individually and/or in combination to facilitate the collection and/or storage of biometric identifiers. By way of a non-limiting example, one implementation of the system 100 may include a collection of one or more biometric identifiers of a minor, though, of course, the systems and methods described herein may be applied to non-minors as well. The collection apparatus 102 may be, by way of a non-limiting embodiment, a pre-fold pamphlet (e.g., a bi-, tri-, or quad-fold pamphlet) to facilitate transportation and storage of the collection apparatus 102. The collection apparatus 102 may be positioned (e.g., in a folded position) in the storage device 106 (such as an envelope) for transit between a distributor (e.g., a vendor, manufacturer, wholesaler, sponsor, etc.) and a user of the collection apparatus 102. Additionally or alternatively, the applicator 104 may also be positioned within the storage device 106 during transit between a distributor and the user of the system 100. During use, the collection apparatus 102 and/or the applicator 104 may be removed from the storage device 106 for collection of the one or more biometric identifiers (as described herein). Subsequent to the collection of the biometric identifier, the collection apparatus 102 may or may not be repositioned within the storage device 106 for long-term storage of the collected biometric identifiers. In some embodiments, the system 100 may also include a sealable, flexible enclosure (e.g., an expandable enclosure) that may be used to receive and store a biometric identifier, such as biological sample. The biological sample may include at least one of a tooth sample, a hair sample, a skin sample, a blood sample, and/or a saliva sample. In some embodiments, the flexible/expandable enclosure may be a sealable bag or baggie.

In some examples, the storage device 106 may include a protective lining configured to help preserve and/or protect a biological sample or biometric identifier (e.g., a DNA sample, such as a direct or touch DNA sample involving a user's saliva or other DNA sample) during storage and/or transportation. For example, the protective lining may include one or more of a moisture-barrier layer (such as a polyethylene or polypropylene film), an oxygen-barrier layer (such as a metallized polyester or aluminum foil layer), a UV-blocking layer (such as a pigmented polymer film), and/or a chemically inert layer (such as a polytetrafluoroethylene (PTFE) or other fluoropolymer layer) configured to limit exposure of the biological sample to humidity, oxygen, and/or light. In some examples, the protective lining may be positioned over a portion of an interior surface of the storage device 106 that is generally aligned with a location of the biological sample collected on the collection apparatus 102 when the collection apparatus 102 is in a folded configuration. For example, the protective lining may be in the form of a panel, patch, or laminate region adhered or bonded to an inner wall of an envelope that directly or indirectly contacts a saliva collection pad or other biological sample region of the collection apparatus 102.

In some examples, the protective lining may extend over substantially all of an interior portion of the storage device 106. For example, the storage device 106 may be formed as a foil-lined envelope or pouch in which an aluminum foil layer, metallized film layer, or polymer-laminate barrier layer covers substantially the entire interior surface of the envelope or pouch to provide environmental protection for any biological sample contained therein.

In some examples, the protective lining may be formed from any suitable material or combination of materials selected to improve protection or enhance the longevity of storage of the biological sample (e.g., a DNA sample, such as a direct DNA sample from a user), and may be selected to avoid materials that could adversely affect the biological sample. For example, the protective lining may include one or more of: (i) a polymer film such as polyethylene, polypropylene, polyethylene terephthalate (PET), polyamide (nylon), or PTFE; (ii) a metal or metalized layer such as aluminum foil or metallized PET; (iii) an absorbent layer, such as silica gel, molecular sieve, or desiccant paper, configured to reduce moisture in the storage device 106; and/or (iv) a pH-neutral, acid-free paper or board layer configured to reduce acidic degradation of DNA. In some embodiments, materials known to adversely affect DNA stability, such as certain plasticizers, PVC, or chlorine-containing materials, may be omitted or reduced.

The system 100 may be used, in at least one implementation, in an identification event to compare against a biometric identifier of an unknown person. For example, a known user's biometric identifier may be deposited and stored on the collection apparatus 102. In the event that the known user goes missing, the deposited biometric identifier may be removed from the collection apparatus 102 and provided to a third party (e.g., law enforcement) to compare against a sample of an unknown individual. In the case of a match between the deposited biometric identifier and the sample, the source individual of the sample is known: the known user.

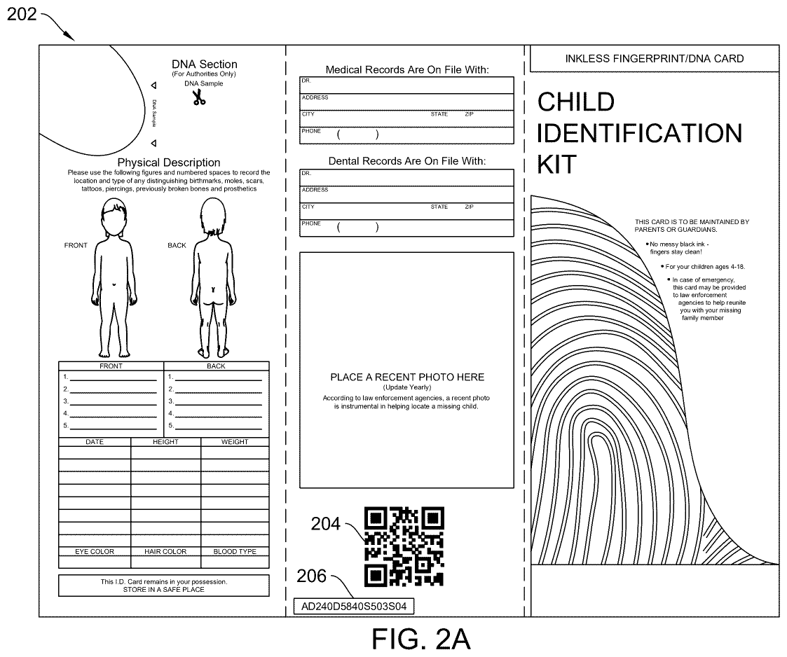

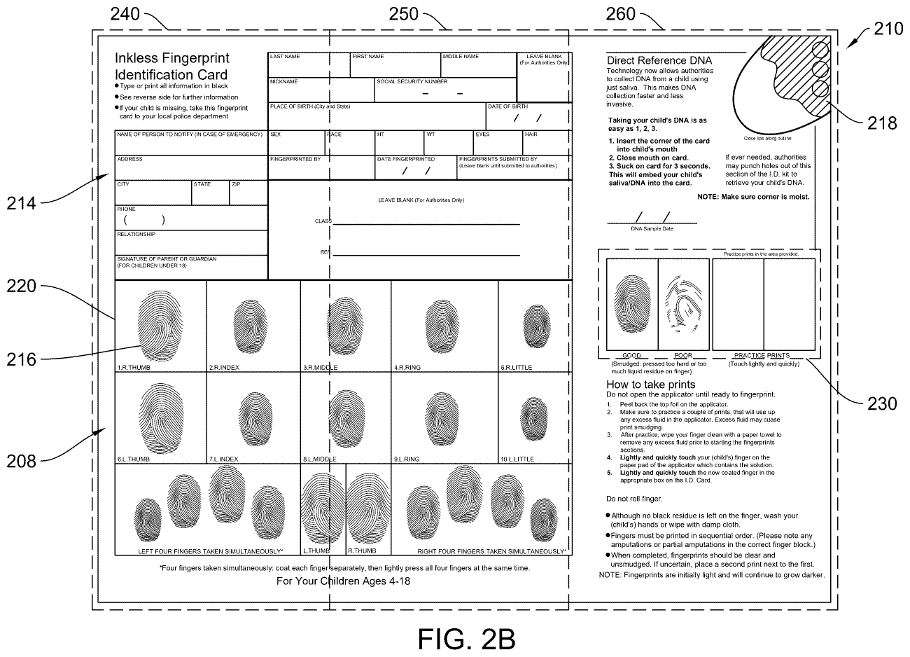

Turning now to FIGS. 2A-2B, a collection apparatus 202 is shown in detail. In some embodiments, the collection apparatus 202 may be substantially similar to the collection apparatus 102 of FIG. 1. The collection apparatus 202 may be any device or combination of devices, materials, systems, and/or apparatuses compatible with the collection of one or more biometric identifiers, such as a fingerprint, a footprint, a handprint, a palm print, an ear impression, a signature, a handwriting analysis, a blood sample, a saliva sample, a skin sample, a hair sample, a fingernail sample, a tooth sample, etc. The collection apparatus 202 may be configured such that biometric identifiers may be deposited thereon/therein and recorded for long-term storage (e.g., 1 year, 5 years, 20 years, 50 years, etc.). In one embodiment, to facilitate at-home collection and storage of biometric identifiers (e.g., to allay privacy concerns with organizational collection of biometric identifiers), the collection apparatus 202 may be of a size and mode compatible with at-home collection and storage processes (as described herein). However, in some embodiments, the collection apparatus 202 may be configured for mass collection of biometric identifiers and not necessarily compatible with at-home collection and storage. For example, the collection apparatus 202 may be configured to collect and store a biometric identifier for a plurality of users. Additionally or alternatively, the collection apparatus 202 may include an area 214 (as shown in FIG. 2B) for biographical input of user whose biometric identifier is collected on the collection apparatus 202. Biographical input may include but is not limited to, name, address, social security number, place of birth, date of birth, emergency contact, sex, race, height, weight, eye color, hair color, phone number, address, etc. FIG. 2A shows a first side of the collection apparatus 202 with additional or alternative locations for user input of user data, such as physical descriptions of the user (e.g., location and type of birthmarks, moles, scars, tattoos, piercings, previously broken bones, and/or prosthetics), location of medical records, location of dental records, etc.

In a non-limiting embodiment, the collection apparatus 202 is a sheet material and/or substrate (e.g., paper). The collection apparatus 102 may be any shape and/or size of paper, such as, for example, A series (e.g., A5, A3), B series(e.g., B4, B5), C series (e.g., C4, C5), letter size (8.5 in.×11 in.), legal size (8.4 in.×14 in.), tabloid size (11 in.×17 in.), and/or executive size (7.25 in.×10.5 in). While the aforementioned sizes are provided for illustrative purposes, they should in no way be considered as limiting. For example, the collection apparatus 202 may be any size and/or shape (e.g., a quadrilateral, a circle, semi-circle, triangle, etc.).

The collection apparatus 202 may be made of paper. Paper provides, in some embodiments, the benefit of allowing fingerprints to be recorded thereon while also being able to absorb biological fluids (e.g., saliva) for long-term storage. While the collection apparatus 202 is described herein as being made of paper (e.g., a collection document or pamphlet) for illustrative purposes, it should be understood that the collection apparatus 202 may be any material that can be used to collect and/or store biometric identifiers. Indeed, in some implementations, the collection apparatus 202 may be comprised of more than one material, each material being particularly suited for collection of one or more types of biometric identifiers. For example, the collection apparatus 202 may be comprised of paper for collection of a first biometric identifier (e.g., a fingerprint such as biometric identifier 216). The collection of a first biometric identifier 216 may be a fingerprint (as shown in FIG. 2B), however, the biometric identifier 216 may be any number of biometric identifiers, including a footprint, handprint, palm print, ear impression, etc. The first area 208 may include, in some implementations, a compound deposited in a layer above some or all of the first area 208. The compound may, in some embodiments, be configured for facilitating the collection of the biometric identifier 216 in a stable and mess-free process. By way of example, the compound may be a chemical coating that interacts with an applicator (e.g., activation solution or agent deposited on the applicator 104 of FIG. 1) such that an interaction area between the chemical coating and applicator is recorded and displayed. For example, if the biometric identifier 216 includes fingerprints, the applicator may be applied to fingertips having fingerprints, and such fingertips may then be placed in contact with the chemical coating, so that the interaction area captures an impression of the fingertips so that the fingerprints are recorded and displayed. In some embodiments, the applicator is an alcohol-based inkless activation solution. For example, neither the chemical coating nor the applicator comprises ink, resulting in a mess-free and easy method for collecting and storing the biometric identifier 216, particularly when used with minors. Though an alcohol-based inkless activation solution is described herein, it should be understood that an ink-based (or other) method for collection of the first biometric identifier 216 may be used.

The first area 208 may include one or more sub-areas, such as collection area 220. The collection area 220 may be demarcated by one or more indicators (e.g., printed lines and/or printed text) to provide an indication of where to place a finger for collection of the biometric identifier 216 and/or an indication of which finger to place for collection. For example, the collection area 220 may be a small, printed box with a label “1. R. THUMB” to indicate that the collection area 220 is for collecting a fingerprint of the user's right thumb. The first area 208 may include a plurality of sub-areas for collecting a plurality of biometric identifiers. In a non-limiting embodiment, the first area 208 includes sub-areas for each finger of the user.

Though the first area 208 is described herein as being paper with a compound applied as a layer on at least a portion of the first area 208, any suitable material for collecting and storing fingerprints may be utilized. For example, additional or alternative materials may include, but are not limited to, adhesive tape to be used with fingerprint powder, plastic film (e.g., a transparent sheet with a coating configured to hold an impression), thermosensitive paper (e.g., paper coated with a thermosensitive material that reacts to pressure and/or heat), metal plates or foils (e.g., Specialized metals like zinc or aluminum to be etched with fingerprints using chemical or heat processes), and/or latent print backing cards.

Additionally or alternatively, for collection and/or storage of a second biometric identifier, such as a biological fluid sample (shown as sample 218), the collection apparatus 202 may include a second area 210. The second area 210 may be paper, just as the first area 208. However, the second area 210 may also include, in some embodiments, a non-toxic material such as a cotton swab, a sponge tip, an absorbent pad, a filter paper, a removable cardstock card, cardboard, and/or a plastic (e.g., polypropylene)/glass collection tube. In a non-limiting implementation of the methods and systems described herein in which the second area 210 is made of paper, the second area 210 is located such that a user may deposit the sample 218 onto the second area 210. By way of example, the second area 210 may be positioned on a corner of the collection apparatus 202. Placement on the corner of the collection apparatus 202 provides the benefit of allowing a user (both adult and child) to place the second area 210 in their mouth and apply the sample 218 to the second area 210 by sucking on it.

In some embodiments, the filter paper may be a porous, absorbent material made from cellulose or synthetic fibers, configured to capture and hold biological fluids like saliva, blood, or other liquid samples of biometric identifiers. The paper's porous structure allows the sample to spread evenly, facilitating efficient drying and non-refrigerated storage. In some embodiments, the filter paper may be treated with chemicals and/or stabilizers to preserve specific biomolecules such as DNA or proteins. These cards may be used collect small volumes of second biometric identifier such as saliva or other biological fluids, which are then dried directly on the card. The dried samples can be stored at room temperature for extended periods, reducing the need for specialized storage conditions. In some embodiments, the removable card is paper or cardstock.

In some embodiments, the collection apparatus 202 may include a flavoring agent embedded into or applied onto at least a portion of the second area 210. This may aid in collection by improving the collection experience of the user. In some embodiments, a flavoring agent aids a young child to suck on at least a portion of the second area 210 to ensure a sufficient amount of saliva is deposited as the sample 218 onto the second area 210. A positioning of the flavoring agent may coincide with an icon and/or graphical representation of the flavoring agent. For example, a strawberry graphic may be applied on or near the second area 210 with the flavoring agent (e.g., with a strawberry flavor) applied on or near the strawberry graphic. The graphic and/or flavoring agent are configured to not contaminate or otherwise appreciably decrease the effectiveness of the sample 218 in positively identifying the user. In a similar manner to the flavoring agent, an olfactory agent may be applied to the collection apparatus 202 (e.g., on and/or near the second area 210). Having the olfactory agent (e.g., scent) applied on the collection apparatus 202 may aid in directing a user to the second area 210 for deposit of the sample 218.

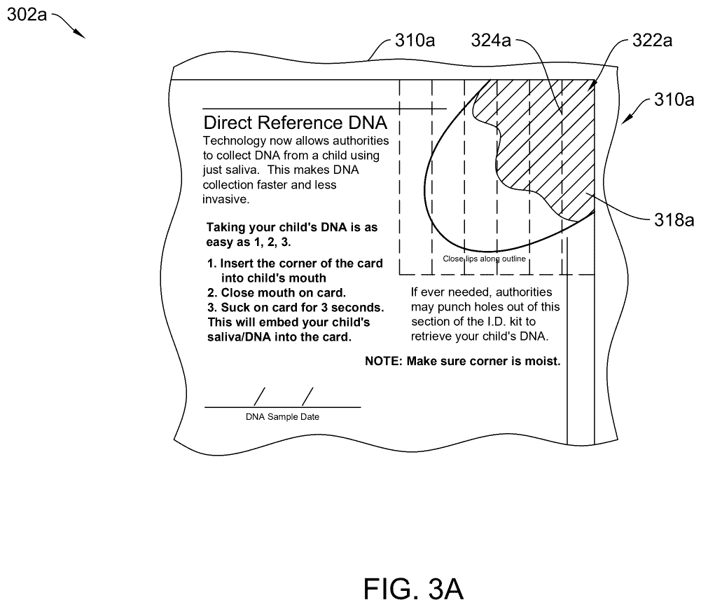

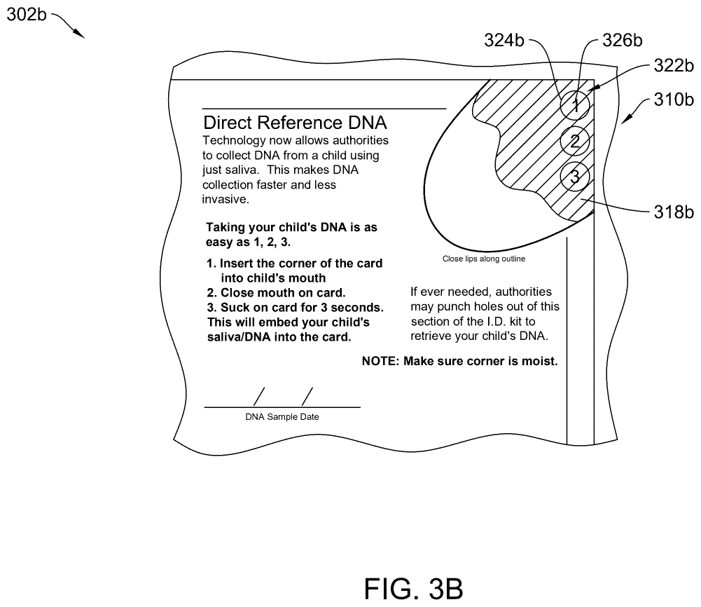

In an illustrative (but non-limiting) embodiment, the second area 210 may be indicated by one or more demarcations. For example, the second area 210 may be surrounded by printed graphics (e.g., a line) to indicate the location for deposit of the sample 218. As shown in FIG. 2B, the location for deposit may be bounded by a printed line in the shape of a tongue. In some embodiments, the second area 210 may include a plurality of sub-areas, as shown in FIGS. 3A-3B.

Turning now briefly to FIG. 3A, a collection apparatus 302a is shown with a second area 310a partitioned into one or more segments 322a. The second area 310a may be substantially similar to the second area 210 of FIG. 2B. The one or more partitioned segments 322a may aid in conservation and/or recordation of the sample 318a. For example, without the partitioned one or more segments 322a, a guardian intending to use the collected sample 318a during an identification event may remove the entire second area 310a to send to a third party for identification of an unknown biological sample using the sample 318a. However, by removing the entirety of the second area 310a, as is likely to be done without visual cues in the form of partitions, the entire sample 318a is put at risk of loss, corruption, and/or contamination. When removed, the sample 318a may be lost in transit to the third party for identification. When removed, the sample 318a may be contaminated with other biometric identifiers (e.g., another's saliva, spit, blood, fingerprints, etc.). When removed, the sample 318a may be destroyed, intentionally or unintentionally. In many instances, the sample 318a may be the only known sample of the user's second biometric identifier. If the sample 318a is completely removed and then lost, contaminated, corrupted, and/or destroyed, there may no longer be any sample with which to identify the user. However, by partitioning the second area 310a into one or more segments 322a, the guardian of the user is guided to remove only the segment 322a, leaving behind a plurality of other segments with the sample 318a embedded therein/thereon. In this way, the sample 318a is preserved for multiple identification processes and/or recordation. In other words, a backup sample is maintained.

The one or more segments 322a may be bounded by one or more demarcation elements 324a. The demarcation element 324a may come in one or more various forms, as shown in FIGS. 3A-3B. For example, as shown in FIG. 3A, the demarcation element 324a may be one or more lines (e.g., a straight line or other shape of a perforation, line, or slit) that partition the second area 310a into one or more segments 322a. The line(s) may be printed onto the second area 310a and may be a solid printed ink line, a dashed ink line (e.g., to indicate a cut line), etc. The demarcation element 324a may alternatively or additionally be a perforated line or slit to facilitate removal of the one or more segments 322a. The demarcation element 324a may be a geometric shape, such as a circle, as shown by a demarcation element 324b of FIG. 3B. As shown in FIG. 3B, a second area 310b may be partitioned into various segments 322b by one or more demarcation elements 324b in the geometric shape (e.g., a circle or any other shape). Demarcation elements (such as the demarcation element 324b) in the shape of a circle may facilitate the independent removal of a specific element, for example, with the use of a punch device (e.g., a hole punch). In some embodiments, the segment 322b may include thereon an identifier 326b (e.g., a number, a barcode, an ID number, a QR code, etc.). FIG. 3B shows the identifier 326b as taking the form of a number (e.g., “1”), however, the identifier 326b may be any authenticating identifier or label. The identifier 326b provides a self-authenticating ID with which to verify the sample 318b. By way of example, in the event that segment 322b is removed and transported to the third party for use in an identification event, the guardian may transmit an indication (e.g., the identifier 326b) of which segment 322b is being provided. Prior to processing the sample 318b, the third party may cross-reference the identifier 326b on the various segments 322b against the identifier indicated by the sender of the segment 322b. It should be understood that an identifier (e.g., the identifier 326b) may be applied to the one or more segments 322a of FIG. 3A as well. The identifier 326b may be located on either side of a collection apparatus 302b within the second area 310b.

In some embodiments, the identifier 326b may be located adjacent or proximate to the segment 322b such that no ink is provided on the segment 322b. In this manner, the segment 322b may be identified when the segment 322b is removed without any possibility of interaction between ink and the segment 322b.

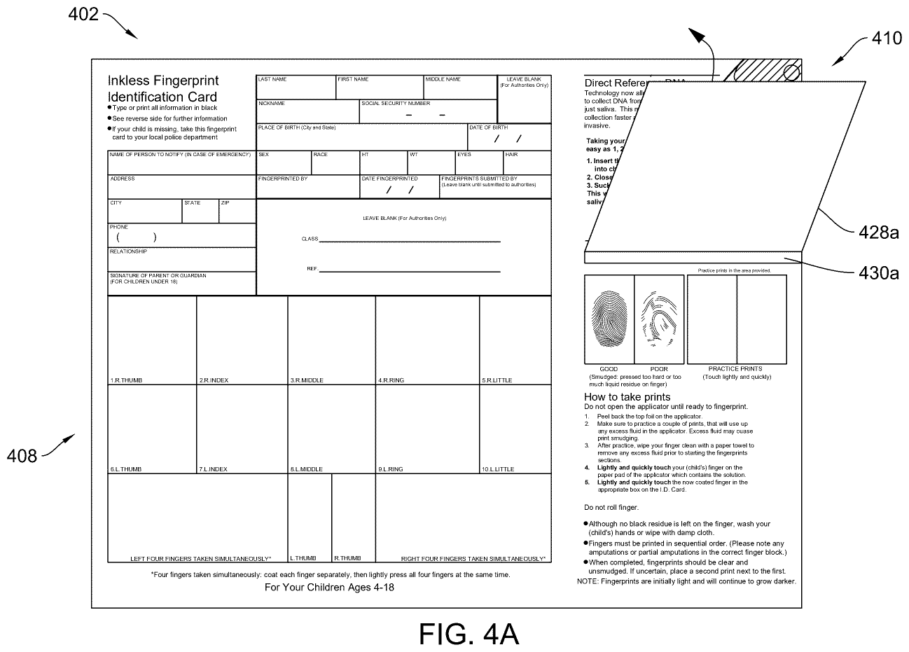

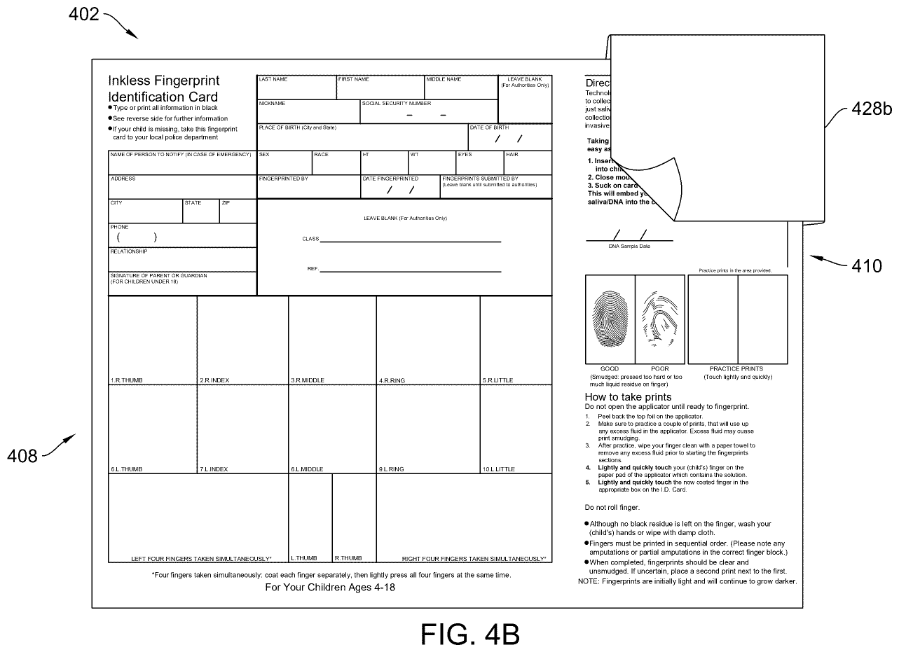

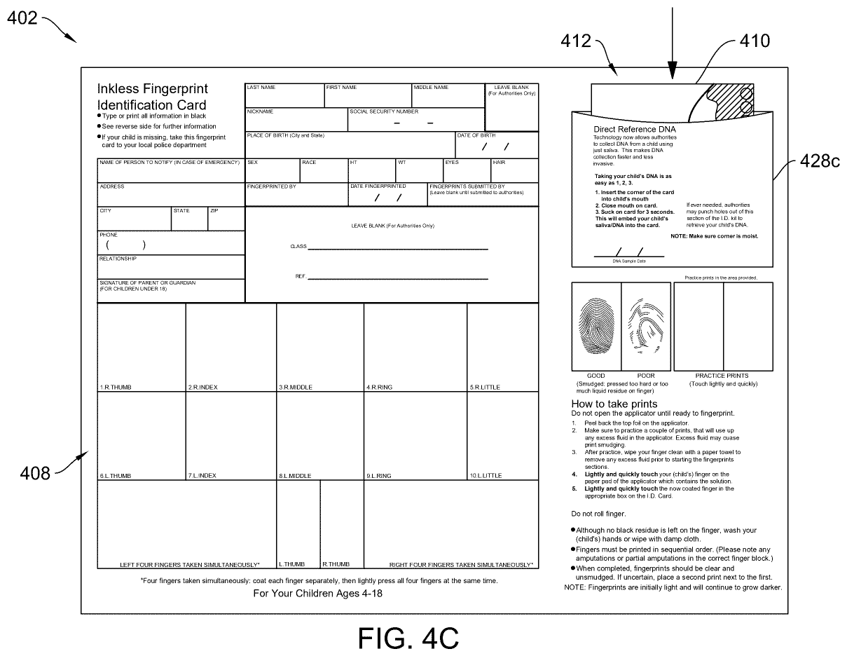

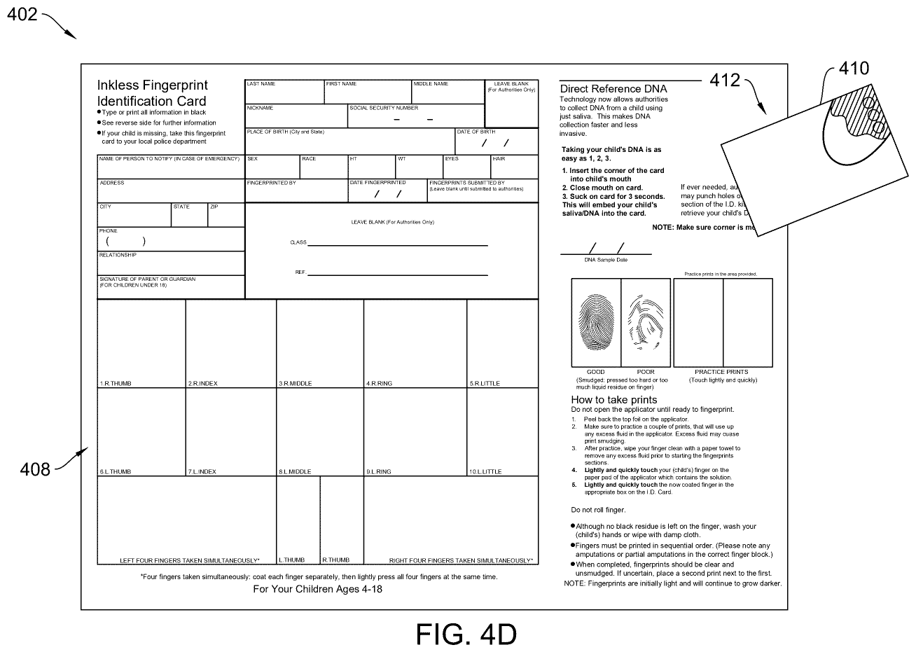

Turning now to FIGS. 4A-4D, various embodiments of protective coverings (e.g., covering 428a, 428b, 428c) for providing a protective barrier over a collection area (e.g., a second area 410) of a collection document (e.g., a collection apparatus 402). As shown in FIG. 4A, the covering 428a may be a flap that is coupled to the collection apparatus 402 and extends from the collection apparatus 402 such that in a first position (e.g., folded open) the flap exposes the second area 410 for collection of the second biometric identifier. The flap may further be adapted such that in a second position (e.g., folded closed) the flap is positioned over the second area 410 (e.g., covers the second area 410). In the second position, the flap may provide a protective layer over the second area 410 to avoid direct contamination of the user's biometric sample (e.g., the sample 218 of FIG. 2B) deposited onto the second area 410 (e.g., contamination via direct contact with another portion of the collection apparatus 402, such as a portion including another biometric identifier, or environmental factors). The flap may be made of one or more of several materials. By way of non-limiting examples, the flap may be made of paper and/or plastic. The flap may be adhered to the collection apparatus 402 at a location 430a at or near the second area 410 such that the flap may cover the second area 410 in the folded configuration. In some embodiments, the flap may be integral to the collection apparatus 402, such that it is not adhered or otherwise externally coupled to the collection apparatus 402.

FIG. 4B illustrates yet another non-limiting embodiment of the protective covering for the second area 410. The covering 428b may be a removable covering configured to be removably coupled to the collection apparatus 402 such that the covering 428b is positioned over the second area 410 to protect the second area 410 from contamination and/or damage. The covering 428b may be made of paper, plastic, and/or other protective material.

FIG. 4C illustrates yet another non-limiting embodiment of the protective covering for the second area 410. The covering 428c may be an expandable enclosure, such as a pocket, that is cooperatively coupled to the collection apparatus 402 and configured to receive at least a portion of the second area 410. In such embodiments, the second area 410 may be decoupled from the collection apparatus 402, such as shown in FIG. 4C. By way of example, the second area 410 may be a removable card 412 (e.g., the size of a business card). In such embodiments, the removable card 412 may be adapted to collect and store a biometric identifier (e.g., a biological fluid or sample). For example, the removable card 412 may be a paper, filter paper, muslin filter, cotton, etc. The removable card 412 may be transported in the covering 428c, removed for biometric identifier sampling/collection, and replaced in the covering 428c for storage while also protecting the sample from direct contamination (e.g., contamination via direct contact with another portion of the collection apparatus 402, such as a portion including another biometric identifier, or environmental factors). In some embodiments, the removable card 412 may be laminated post-collection to seal in the collected sample.

In some embodiments, the removable card first area 410 (or other removable card) shown in FIG. 4C may be used to collect a biometric identifier of a second user, such as a guardian or caregiver of the main user of the collection apparatus 402. By way of example, the main user may be a minor and the guardian is the minor's parent. The minor's biometric identifiers may be collected on the collection apparatus 402 at the first area 408 and/or the second area 410 (when the second area 410 is part of the collection apparatus 402, such as shown in FIG. 2B). The guardian's biometric identifier (e.g., saliva) is then collected on the removable second area 410 (as shown in FIG. 4C) and placed with the collection apparatus 402, such as in the covering 428c. This minor-guardian identifier collection may be used to deter unauthorized possession of the minor by a non-guardian. Likewise, the collection apparatus 402 may include additional areas (e.g., a third area 230, as shown in FIG. 2B) for collection of an additional or alternative biometric identifier of the guardian (e.g., one or more fingerprints). While FIG. 2B illustrates the third area 230 as a location for practicing collection of the first biometric identifier, it may alternatively or additionally be used to collect a biometric identifier of the second user (e.g., the guardian).

As shown in FIG. 4D, a system 400, which may be substantially similar to the system 100 of FIG. 1) may include the removable card 412 (e.g., as shown in FIG. 4C) on which the second area 410 is positioned. However, in an embodiment illustrated in FIG. 4D, the collection apparatus 402 need not include the covering 428c of FIG. 4C. In such embodiments, the removable card 412 is freely movable from the collection apparatus 402 and may be positioned within the folded collection apparatus 402 during transport/storage. The removable card 412 may also be separated from the collection apparatus 402 to be sent to an organization or agency in an effort to identify a missing person. For example, the removable card 412 (of a portion of the removable card 412) may be transmitted to a law enforcement agency for DNA sampling and analysis to compare the biometric identifier (e.g., DNA, such as direct or touch DNA) on the removable card 412 against a sample of collected DNA or to include in a database of biometric identifiers to be used in a later comparison.

The various coverings 428a, 428b, 428c may, in various embodiments, prevent contamination of the collected sample on the second area 410 by one or more contaminants. For example, the covering 428a, 428b, 428c prevent the second area 410 from coming into contact with other portions of the collection apparatus 402, such as a first area 408. The first area 408 may be substantially similar to the first area 208 of FIG. 2B. As described herein, the first area 408 may include a chemical coating compound on at least a portion of the second area 410. In some embodiments, this chemical coating compound (or the applicator used to activate the chemical coating compound during fingerprint collection) may interact with the second biometric identifier sample if either the chemical coating compound, the applicator, or the combined compound-applicator mixture comes in contact with biometric identifier. As such, preventing contact between the first area 408 and the second area 410 (or at least in some embodiments, the biometric identifier sample deposited onto the second area 410) may provide a benefit of reducing the potential of contaminated samples. While the interaction between the first area 408 and the second area 410 may, in some implementations, not reduce the efficacy of the sample, separation of the two areas may nevertheless provide one or more benefits. By way of one non-limiting example, the user of the collection apparatus 402 may have concerns of depositing a sample on the second area 410 if the second area 410 has been in contact with the first area 408, which may or may not have a chemical compound layered thereon. Applying a protective covering over the first area 408 and/or the second area 410 prevents contact between the two areas. However, placement of the first area 408 and/or the second area 410 may also prevent contact between the two areas.

Returning now to FIG. 2B. As described above, the collection apparatus 202 may be embodied in a pre-folded paper pamphlet, such as a tri-fold pamphlet. In some embodiments, the first area 208 may be in a location relative to the second area 210 such that in a first configuration of the collection apparatus 202 (e.g., unfolded in the open position), the first area 208 is presented to the user for collection of the first biometric identifier (e.g., one or more fingerprints), yet in a second configuration (e.g., closed), the first area 208 and the second area 210 are not in contact with one another. For example, in the implementation in which the collection apparatus 202 is a pre-folded paper pamphlet (e.g., a tri-fold pamphlet divided substantially equally into three panels, shown in FIG. 2B as a first panel 240, a second panel 250, and a third panel 260), the first area 208 may be positioned on a lower portion of the first panel 240, the second panel 250, and/or a third panel 260. The second area 210 may be located on an upper portion of the first panel 240, the second panel 250, and/or the third panel 260. In some embodiments, the lower portion and the upper portion do not overlap, such that the first area 208 and the second area 210 do not come into contact with one another when the collection apparatus 202 is in the second configuration (e.g., a folded position). Further, when in the first configuration (e.g., an opened position), the first area 208 and/or the second area 210 may be presented to the user for collection of one or more of the biometric identifiers. In this manner, collected biometric identifiers (or the layer of chemical compound on the first area 208) do not cross-contaminate each other, but are nonetheless protected from the surrounding environment during storage.

Though the first area 208 is described and illustrated as being positioned at a location on the lower portion of the collection apparatus 202, and the second area 210 is described and illustrated as being positioned at a location on the upper portion of the collection apparatus 202, it should be understood that these are for illustrative purposes only. The first area 208 and the second area 210 may be placed in any position relative to each other such that the two areas do not come into contact with each other when the collection apparatus 202 is in its designed configurations (e.g., pre-folded and opened).

An additional or alternative benefit, in at least one implementation, of storing the collection apparatus 202 in the folded configuration is that the samples of biometric identifiers (e.g., fingerprints and/or fluid sample such as saliva) are protected from light. The collection apparatus 202 may be configured such that in the folded configuration, the first area 208 and/or the second area 210 are folded into an interior of the collection apparatus 202. In this way, the biometric identifiers are protected from potential light degradation over time. Likewise, being positioned in the interior of the collection apparatus 202 in the folded configuration allows the biometric identifiers to be stored with a reduced risk of contamination from the environment. In other words, the biometric identifiers are protected from the external environment by being folded into the interior of the collection apparatus 202.

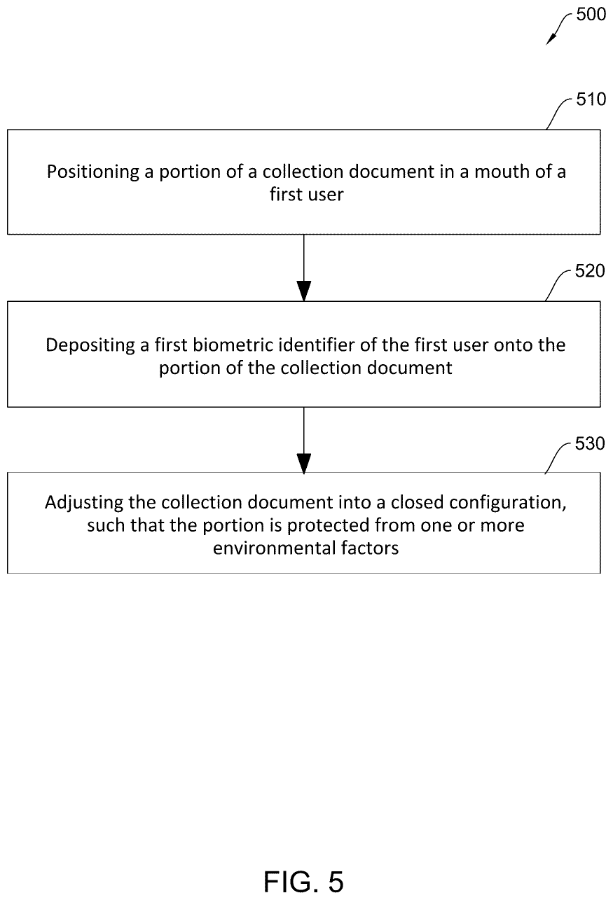

Turning to FIG. 5, a flow diagram of a method 500 for collecting and storing one or more biometric identifiers on a collection document is illustrated. Although FIG. 5 illustrates the steps 510, 520, and 520 in a specific order, it should be understood that the method of FIG. 5 may include more, less, or different steps than those illustrated in FIG. 5 without departing from the scope of the present disclosure. Additionally or alternatively, the method may be performed in any order. The method may be performed by a user whose biometric identifier is being collected and/or by a second user whose biometric identifier is not being collected. Likewise, providing instructions for performing the method of FIG. 5 falls within the systems and methods described herein.

At step 510, the method 500 may include positioning a portion of a collection document in a mouth of a first user, the collection document having a first panel (e.g., the first panel 240 of FIG. 2B, the second panel 250 of FIG. 2B, and/or the third panel 260 of FIG. 2B), a second panel (e.g., the first panel 240 of FIG. 2B, the second panel 250 of FIG. 2B, and/or the third panel 260 of FIG. 2B), and a third panel (e.g., the first panel 240 of FIG. 2B, the second panel 250 of FIG. 2B, and/or the third panel 260 of FIG. 2B), and wherein the portion is located at least in part on the third panel. In some examples, the collection document can have any number of panels, such as two panels, three panels, four panels, or five panels, among others. The collection document may be substantially similar to the collection apparatus 102, 202, 302, and 402. In an implementation, the portion is positioned/located on a corner of the collection document, as shown by the second area 210 in FIG. 2B. While the portion is illustrated in various figures and descriptions herein as being located on a corner, it is understood that the portion may be located anywhere on the collection document, including along an edge not on a corner or distanced from the edge of the document (e.g., in a middle area of the collection document). In embodiments in which the portion is located at a position nonadjacent to the edge, the method may include positioning a user's tongue on the portion to deposit a biometric identifier sample onto the portion, such as described at step 520. Step 520 of the method may include depositing a first biometric identifier of the first user onto the portion of the collection document while the collection document is positioned within the mouth of the first user. Depositing the first biometric identifier onto the portion may include sucking on the portion for a threshold time (e.g., 1 second, 2 second, 3 seconds, 5 seconds, etc.). While sucking on the portion is provided for exemplary purposes, it should be understood that alternative or additional methods of depositing the first biometric identifier may be used, such as swabbing, expectorating, scraping, touching, etc.

At step 530, of the method 500 may include adjusting the collection document into a closed configuration. In at least one implementation, adjustment of the collection document may include folding the third panel such that a first face of the portion of the third panel upon which the first biometric identifier is deposited is positionally opposing a first unprinted section or non-identifier including section of a second face of the second panel.

The adjustment may additionally or alternatively include folding the first panel such that a second unprinted section of a third face of the first panel is positionally opposing a fourth face of the portion of the third panel upon which the first biometric identifier is deposited, wherein when the collection document is in the closed configuration, the portion is positioned between the first panel and the second panel such that the portion is protected from one or more environmental factors. Environmental factors may include, but are not limited to, light, moisture, heat, humidity, contaminants, dust, fingerprints, liquids, other users, etc. By folding the collection document (which may be, in some embodiments, a pamphlet) in such a way that the collected biometric identifier sample is positioned within the pamphlet when in the closed configuration, the collected sample may be protected from various potentially destructive and/or contaminating substances external to the collection document.

The methods described herein may also provide for a configuration in which the collected biometric identifier sample is positioned within the pamphlet to be protected from potential contaminating substances integral to the collection document. For example, the placement of various portions and areas of the collection document may be configured such that the sample is not touching any printed text and/or graphics when in the closed configuration. For example, the areas of the first and second panels that the collected sample is in contact with when in the closed configuration may not contain printed data (although, the collection apparatus shown in FIGS. 1-4C shows one or more embodiments in which the collected sample may be in contact with printed text or graphics). Additionally or alternatively, in some embodiments, the areas of the first and second panel that the collected sample is in contact with when in the closed configuration may contain printed text or graphics, but do not contain areas for collection of a second biometric identifier, such as the first user's fingerprints or a second user's biofluid sample (e.g., a guardian's saliva). Such embodiments are illustrated in FIGS. 1-4C. Likewise, in embodiments, in which a second user (e.g., the guardian) deposits a second biometric identifier sample onto the collection document on a demarcated second portion, the location of the demarcated second portion may be positioned such that in the closed configuration, at least a portion of the demarcated second portion may not be in contact with other biometric identifiers (e.g., of themselves or another user) and/or printed text or graphics. In some examples, when the collection document is in the closed configuration, an additional portion of material (e.g., a pouch, separating card, etc.) may be placed between the collected biometric identifier sample and other portions of the collection document (e.g., first panel, second panel, etc.) to provide further protection for the collected biometric identifier.

Returning to FIG. 2A, the collection apparatus 202 may include disposed thereon an identifier shown as identifier 204. The identifier 204 may be an identifier (e.g., a quick response code, a uniform resource locator, a barcode, a data matrix code, an RFID chip, an NFC chip, an Aztec code, an augmented reality marker, etc.) that may be used to navigate a user of the collection apparatus 202 to a page (e.g., an electronic webpage) associated with the collection apparatus 202 or an entity associated with the collection apparatus 202. For example, in at least one embodiment, the identifier 204 is a QR code which is optically scanned by an electronic device to cause the electronic device to electronically navigate to a webpage or application associated with the collection apparatus 202. The webpage or application may allow the user of the electronic device to input data into various input fields and to optionally store the inputted data in a database. The user may provide biographic information such as a name, address, social security number, place of birth, date of birth, emergency contact, sex, race, height, weight, eye color, hair color, phone number, address, fingerprints, etc. The data may be inputted manually through text input and/or, optionally, inputted automatically by capturing an image of the collection apparatus 202.

When capturing an image (e.g., with the electronic device such as a cellular device) of the collection apparatus 202, the electronic device may prompt the user to align a field of view of a camera of the electronic device with the collection apparatus 202. For example, the electronic device may present for display an outline of the collection apparatus 202 overlayed on a capture screen showing the field of view of the electronic device which may be used to orient the electronic device such that the collection apparatus 202 is aligned with the outline of the collection apparatus 202. In some embodiments, the electronic device automatically captures a picture of the collection apparatus 202 upon alignment of the borders of the collection apparatus 202 with the outline overlaid on the display of the electronic device. In some embodiments, the user manually captures the image through the use of an interactive graphical component, such as a digital button. In some embodiments, the electronic device prompts the user to flip the collection apparatus 202 to the other side (e.g., as shown in FIG. 2B) and the same process may be repeated for image capture of the other side of the collection apparatus 202.

One or more processors of the electronic device (or one or more processors communicably coupled to the electronic device) may perform an optical character recognition of any text written on the collection apparatus 202. The recognized text (e.g., in specific fields of the collection apparatus 202) is extracted and inputted into a storage database. In some embodiments, the user must affirmatively select to input the data to the storage database through, for example, acceptance of a storage/privacy policy describing the use of the data when stored in the storage database. In some embodiments, the storage database is a law enforcement database. In some embodiments, the data may be stored privately (e.g., with access only to the user) with the option to later upload the data to a storage database associated with law enforcement, such as when an individual goes missing. In some embodiments, the data is uploaded to the storage database in a Criminal-Justice-Information-Services-ready format.

The electronic webpage of which the identifier 204 navigates the electronic device may also provide instructions to the user on steps to take upon an individual (e.g., a child) going missing. This may include contacting law enforcement, uploading data on the collection apparatus 202 to the storage database, scheduling a meeting with law enforcement, etc. The electronic webpage may allow the user to report the individual missing, which may optionally cause one or more of the steps above to be automatically executed. In some embodiments, the identifier 204 is used by law enforcement to collect the biographical information related to the missing individual. In some embodiments, each collection apparatus 202 has a unique identifier 206 (e.g., a serial alphanumeric identifier) which may be used to identify the individual collection apparatus 202 and/or samples collected thereon. This unique identifier may be automatically collected during the optical capture of the information on the collection apparatus 202 and used to associate the collected data with a specific user or collection apparatus 202.

In some examples, the identifier 204 can include an embedded serial number, code, or other identifier that is unique to the collection apparatus 202 provided to a particular user and can be used to provide access to a webpage configured for the user or the collection apparatus 202. In some examples, the identifier 204 can provide access to a general webpage used for entering information for multiple collection apparatuses provided to multiple users. In some examples, the identifier 204 can be printed on a front side of the collection apparatus 202, and in other examples, the identifier 204 can be printed on a backside of the collection apparatus 202. In some examples, the identifier 204 can be covered by a scratch-off area. The scratch-off area can refer to a portion of the collection apparatus 202 formed from a printable material or coating such as a latex-based scratch-off ink, a polymer-based scratch-off coating, or a foil-type scratch-off laminate configured to be removed by rubbing or scraping (e.g., with a fingernail or coin). In some examples, the identifier 204 may be concealed from view until the user or a second user intentionally removes at least a portion of the scratch-off layer. In some examples, the identifier 204 or another indicator on the collection apparatus 202 may include a name of the user. In some examples, the collection apparatus 202 can include a uniform resource locator (URL) that links to the webpage and/or a phone number to call to confirm completion of collection of biometric identifiers via the collection apparatus 202. These and other features of the collection apparatus 202 and/or identifier 204 can improve security by preventing fake or counterfeit collection apparatuses while maintaining user privacy.

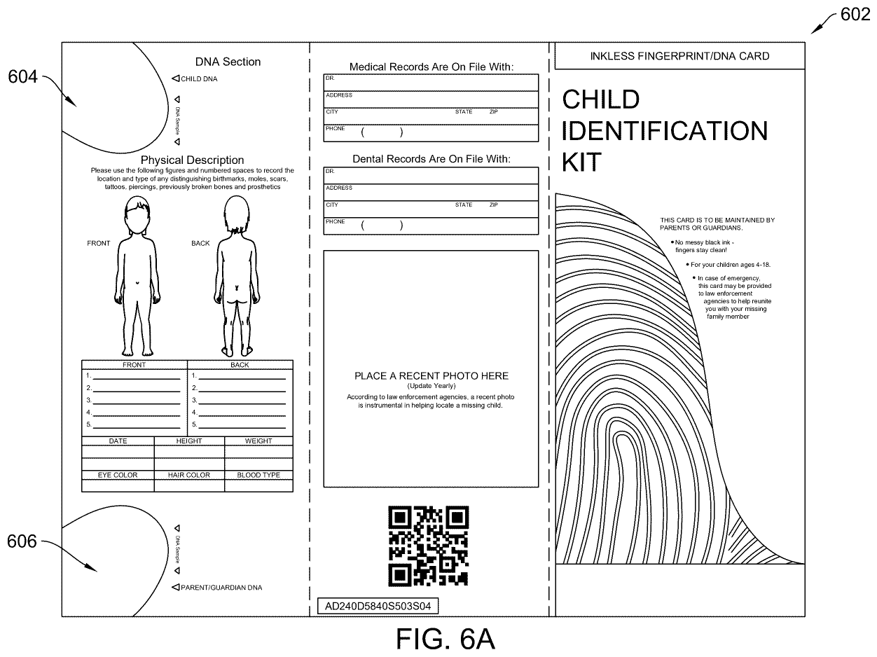

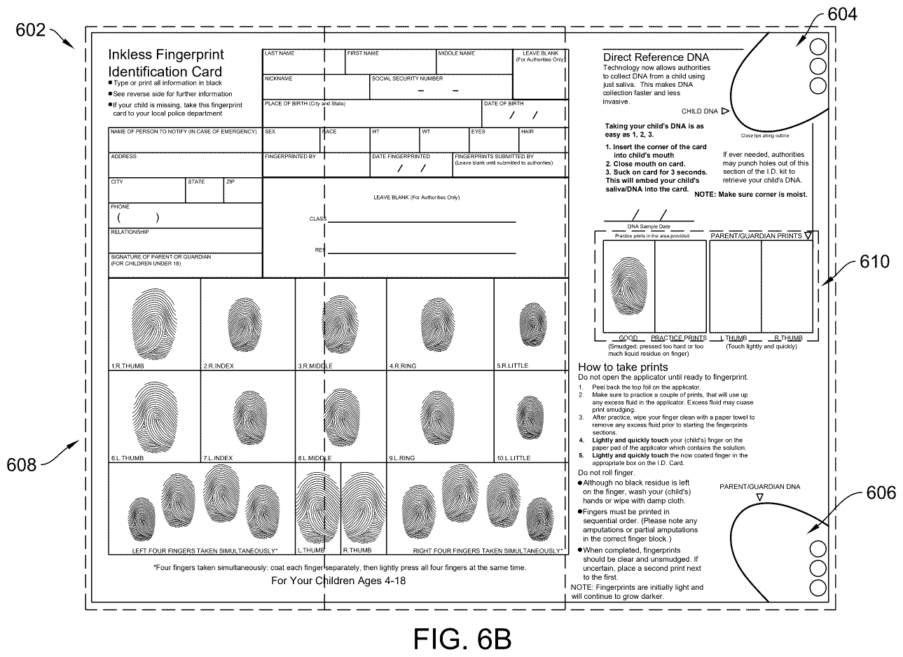

Referring to FIG. 6A, a first side of a collection document 602 for collecting one or more biometric identifiers is shown. In some examples, the collection document 602 can include an area (e.g., second area 604) for a second collection of a second biometric identifier that includes DNA of the user (e.g., a direct or touch DNA sample, such as a saliva sample of the child). In some examples, the collection document 602 can include another area (e.g., fourth area 606) for collection of a fourth biometric identifier that includes DNA of the second user (e.g., a direct or touch DNA sample, such as a saliva sample of a parent or guardian of the child). The collection document 602 can also include one or more labels indicating which area corresponds to a particular a user (e.g., the second area 604 can be identified by a label indicating the second area 604 is configured to collect DNA, such as direct or touch DNA, from a child, the fourth area 606 can be identified by a label indicating the fourth area 606 is configured to collect DNA, such as direct or touch DNA, from a parent/guardian of the child). In some examples, the first or second area and accompanying labels can be positioned on the collection document 602 in visual proximity to visual displays or other information corresponding to a respective one of the user or the second user. For example, the second area 604 and associated label can be positioned near or otherwise within visual proximity of an anatomical diagram of a child (e.g., the distance between the diagram and the second area 604 can be less than the distance between the diagram and the fourth area 606).

Referring to FIG. 6B, a second side of the collection document 602 for collecting one or more biometric identifiers is shown. In some examples, the second area 604 can be positioned on the collection document 602 such that, when the collection document 602 is in a folded or closed configured, that second area 604 does not contact a first area 608 configured to collect another biometric identifier (e.g., fingerprint impressions) of the user. For example, the positioning of the second area 604 and first area 608 can be configured such that DNA and fingerprint impressions of the child are protected from cross-contamination. In some examples, the collection document 602 can include a third area 610, which can be configured to collect a third biometric identifier of the second user (e.g., fingerprint impressions of the parent or guardian). In some examples, the third area 610 can be positioned on the collection document 602 such that, in a folded or closed configuration, at least a portion of the third area 610 does not contact the first area 608.

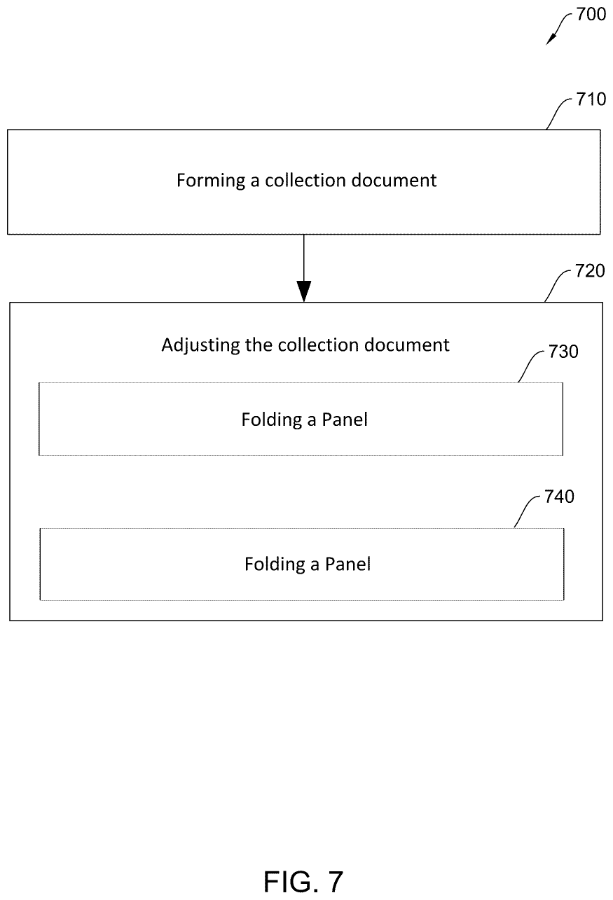

Referring to FIG. 7, a flow diagram of a method 700 for manufacturing a collection document for collecting and storing one or more biometric identifiers is shown. At step 710, the method 700 may include forming a collection document. In some examples, forming the collection document can include printing one or more regions for receiving biometric identifiers (e.g., fingerprint regions, palm regions, toeprint regions, or facial contact regions), printing textual instructions or diagrams for a user or second user, printing one or more identifiers (e.g., a serial number, bar code, QR code, etc.), and/or applying one or more coatings or layers (e.g., a layer of a compound configured to receive an impression of a biometric identifier, a protective varnish, hydrophobic or hydrophilic coating, a scratch-off area, etc.).

In some examples, forming the collection document can include forming a portion of the collection document configured to be positioned in a mouth of a first user. For example, forming the collection document can include providing, marking, or otherwise defining a saliva collection area or another oral-contact region that is sized, shaped, or otherwise configured (e.g., via treatment with agents or other properties) to be placed at least partially in the mouth of the first user (e.g., child). In some examples, the collection document can have any number of panels, such as a first panel, a second panel, and a third panel. For example, the collection document can be formed from a foldable substrate (e.g., paper, cardstock, cardboard, or a polymer sheet) that is creased or scored to define a tri-fold pamphlet, such that the first, second, and third panels are created by folding along one or more fold lines. In some examples, the portion configured to be positioned in the mouth of the first user can be located at least in part on the third panel, and the portion can be configured to collect a first biometric identifier of the first user while the collection document is positioned within the mouth of the first user.

In some examples, forming the collection document can include applying a layer of a compound to one or more other portions of the collection document configured to collect an impression of a second biometric identifier (e.g., fingerprint) of the first user or an impression of a third biometric identifier (e.g., fingerprint) of a second user. In some examples, forming the collection document can include forming an additional portion of the collection document configured to collect a fourth biometric identifier including DNA of the second user (e.g., direct or touch DNA, such as a saliva sample of the second user).

At step 720, the method 700 can include performing an adjustment or folding operation, or providing or forming the collection document for one or more adjustments. The one or more adjustments can include folding or otherwise adjusting the collection document from an open configuration to a closed configuration. For example, the method 700 can include providing demarcations or other indications of the collection document (e.g., printed lines, perforations, etc.) to facilitate adjusting the collection document from an open to a closed configuration. In some examples, the method 700 can include folding, scoring, or otherwise adjusting the collection document from an unfolded (e.g., open) to folded (e.g., closed) configuration. At step 730, the method 700 can include performing an adjustment or folding operation, or providing or forming the collection document for one or more adjustments. In some examples, the method 700 can include folding the third panel of the collection document such that a first face of the third panel of the collection document is positionally opposing a first face of the second panel. At step 740, the method 700 can include performing an adjustment or folding operation, or providing or forming the collection document for one or more adjustments. For example, the method 700 can include folding the first panel such that a first face of the first panel is positionally opposing a second face of the third panel. For example, the first biometric identifier that is deposited on the third panel can be positionally opposition a first section of the first face of the second panel that does not include a biometric identifier, such that the third panel is positioned between the first panel and the second panel while in the closed configuration. For example, printed information or biometric collection areas of the collection document can be arranged or positioned such that the portion or area of the collection document configured to collect DNA of the first user, such as direct DNA, is protected from contamination from printed information or other biometric collection areas of the collection document (e.g., protected from environmental factors). For example, the portion of the collection document configured to capture the first biometric identifier can be formed such that, when the collection document is in the closed configuration, the portion is positioned between the first panel and the second panel such that the portion is protected from other collected biometric identifiers (e.g., fingerprints) or environmental factors.

In some examples, the one or more environmental factors can include one or more of light, moisture, heat, humidity, contaminants, and dust. In some examples, the collection document can be formed such that the collection document is configured to be adjusted into an open configuration. For example, the collection document can be printed, folded, or otherwise formed such that that the portion of the third panel is positioned outside of the first panel and the second panel when the collection document is in the closed configuration. In some examples, the collection document includes a second portion configured to collect a second biometric identifier. For example, the collection document can be printed, folded, or otherwise formed such that the second portion is positioned at least partially upon the second panel such that the second biometric identifier is not in contact with the first biometric identifier when the collection document is in the closed configuration. In some examples, the collection document includes a second portion where a second biometric identifier may be collected. The second portion can be positioned on the third panel such that the third panel is positioned between the first panel and the second panel while in the closed configuration such that the second portion of the third panel is at least partially protected (e.g., from contamination or environmental factors).

In some examples, forming the collection document can include selecting a base material or substrate (such as paper, cardstock, cardboard, or a polymer sheet), cutting the material to a desired size and outline (e.g., with a die-cutting or slitting process), and then creasing or scoring the material along predetermined fold lines to define the first panel, the second panel, and the third panel. Biometric collection regions on one or more of the panels can be manufactured by printing outlines, labels, or graphics that visually indicate where a fingerprint or mouth of a user is to be placed, and applying any coatings or layers to facilitate collection (e.g., a layer of a compound configured to receive an impression of a biometric identifier, one or more scented or flavored portions configured to assist in DNA collection, etc.). Instructional content can be manufactured by printing text, diagrams, and icons on one or more panels using printing methods (e.g., offset, digital, or flexographic printing), while identifiers (such as serial numbers, bar codes, or QR codes) can be manufactured by generating identifiers using a computing program and printing or encoding the identifiers in designated areas of the collection area. Unprinted sections or low-printing sections used for protecting collection regions in the closed configuration can be manufactured by configuring printer instructions such that the printer omits ink in those areas. In some examples, coatings or linings can be added to selected areas of the collection document or associated storage devices to improve durability, moisture resistance, or contamination control.

As utilized herein, the terms “approximately,” “about,” “substantially,” and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the disclosure as recited in the appended claims.

It should be noted that the term “exemplary” and variations thereof, as used herein to describe various embodiments, are intended to indicate that such embodiments are possible examples, representations, or illustrations of possible embodiments (and such terms are not intended to connote that such embodiments are necessarily extraordinary or superlative examples).

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

The term “or,” as used herein, is used in its inclusive sense (and not in its exclusive sense) so that when used to connect a list of elements, the term “or” means one, some, or all of the elements in the list. Conjunctive language such as the phrase “at least one of X, Y, and Z,” unless specifically stated otherwise, is understood to convey that an element may be either X, Y, Z; X and Y; X and Z; Y and Z; or X, Y, and Z (i.e., any combination of X, Y, and Z). Thus, such conjunctive language is not generally intended to imply that certain embodiments require at least one of X, at least one of Y, and at least one of Z to each be present, unless otherwise indicated.

References herein to the positions of elements (e.g., “top,” “bottom,” “above,” “below”) are merely used to describe the orientation of various elements in the FIGURES. It should be noted that the orientation of various elements may differ according to other exemplary embodiments, and that such variations are intended to be encompassed by the present disclosure.

The hardware and data processing components used to implement the various processes, operations, illustrative logics, logical blocks, modules and circuits described in connection with the embodiments disclosed herein may be implemented or performed with a general purpose single-or multi-chip processor, a digital signal processor (DSP), an application specific integrated circuit (ASIC), a field programmable gate array (FPGA), or other programmable logic device, discrete gate or transistor logic, discrete hardware components, or any combination thereof designed to perform the functions described herein. A general purpose processor may be a microprocessor, or, any conventional processor, controller, microcontroller, or state machine. A processor also may be implemented as a combination of computing devices, such as a combination of a DSP and a microprocessor, a plurality of microprocessors, one or more microprocessors in conjunction with a DSP core, or any other such configuration. In some embodiments, particular processes and methods may be performed by circuitry that is specific to a given function. The memory (e.g., memory, memory unit, storage device) may include one or more devices (e.g., RAM, ROM, Flash memory, hard disk storage) for storing data and/or computer code for completing or facilitating the various processes, layers and modules described in the present disclosure. The memory may be or include volatile memory or non-volatile memory, and may include database components, object code components, script components, or any other type of information structure for supporting the various activities and information structures described in the present disclosure. According to an exemplary embodiment, the memory is communicably connected to the processor via a processing circuit and includes computer code for executing (e.g., by the processing circuit or the processor) the one or more processes described herein.

The present disclosure contemplates methods, systems and program products on any machine-readable media for accomplishing various operations. The embodiments of the present disclosure may be implemented using existing computer processors, or by a special purpose computer processor for an appropriate system, incorporated for this or another purpose, or by a hardwired system. Embodiments within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. Combinations of the above are also included within the scope of machine-readable media. Machine-executable instructions include, for example, instructions and data which cause a general-purpose computer, special purpose computer, or special purpose processing machines to perform a certain function or group of functions.

Although the figures and description may illustrate a specific order of method steps, the order of such steps may differ from what is depicted and described, unless specified differently above. Also, two or more steps may be performed concurrently or with partial concurrence, unless specified differently above. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

What is claimed is:

1. A system comprising:
a collection apparatus comprising:
a first area for a first collection of a first biometric identifier of a user, the first area positioned at a first location of the collection apparatus;
a compound applied as a layer to at least a first portion of the first area;
a second area for a second collection of a second biometric identifier that includes DNA of the user;
wherein the second area is positioned at a second location of the collection apparatus, the second location of the collection apparatus positioned such that in a first configuration of the collection apparatus, the second area is presented for the second collection of the second biometric identifier that includes the DNA of the user;
wherein the second location of the collection apparatus is positioned such that in a second configuration of the collection apparatus, the second area is not in contact with the first area; and
an applicator that, when in contact with a portion of the layer of the compound, interacts with the portion of the layer of the compound such that an impression of the first biometric identifier of the user is captured.

2. The system of claim 1, wherein the collection apparatus further comprises a third area for collection of a third biometric identifier of a second user.

3. The system of claim 2, wherein the collection apparatus further comprises a fourth area for collection of a fourth biometric identifier that includes DNA of the second user.

4. The system of claim 2, wherein the second user is a guardian or parent of the user.

5. The system of claim 2, wherein the third area is positioned on the collection apparatus at a third location of the collection apparatus;
wherein the third location of the collection apparatus is positioned such that in the second configuration at least a portion of the third area is not in contact with the first area.

6. The system of claim 1, further comprising a covering configured to be applied to at least one or more of the first area or the second area such that the covering prevents direct contamination of at least the second biometric identifier with another biometric identifier.

7. The system of claim 6, wherein the covering is an expandable enclosure that is coupled to the collection apparatus and configured to receive at least a second portion of the second area.

8. The system of claim 6, wherein the covering is a flap coupled to and extending from the collection apparatus such that in a first position the flap exposes the second area for collection of the second biometric identifier and in a second position the flap covers the second area.

9. The system of claim 6, wherein the covering is at least partially removeably coupled to the collection apparatus such that when the covering is coupled to the collection apparatus it covers at least one of a portion of the first area or the second area.

10. The system of claim 1, wherein the second area is partitioned into a plurality of segments.

11. The system of claim 10, wherein the plurality of segments are demarcated by one or more demarcation elements.

12. The system of claim 11, wherein the one or more demarcation elements include at least one of a perforation, a line, and a slit.

13. The system of claim 11, wherein the one or more demarcation elements include a geometric shape for removal from the second area by a punch.

14. The system of claim 1, wherein the collection apparatus includes an expandable enclosure for collection of a fifth biometric identifier of the user.

15. The system of claim 14, wherein the fifth biometric identifier is a biological sample, including at least one of a tooth sample, a hair sample, a skin sample, a blood sample, or a saliva sample.

16. The system of claim 1, wherein the collection apparatus is pre-folded at one or more locations.

17. The system of claim 16, wherein the collection apparatus is pre-folded at two locations such that the collection apparatus is divided into three panels.

18. The system of claim 17, wherein the first area is positioned on at least a third portion of a first panel and a second panel, and the second area is positioned on at least a fifth portion of a third panel.

19. The system of claim 1, wherein at least a portion of the second area has provided thereon a flavoring agent.

20. The system of claim 1, wherein the applicator is an alcohol-based inkless activation solution.

21. The system of claim 1, wherein the collection apparatus includes an identifier for an electronic page for inputting data associated with the collection apparatus.

22. The system of claim 21, wherein the electronic page displays an interactive graphical component that, when selected, stores the inputted data to a storage database.

23. The system of claim 1, further comprising a storage device configured to store the collection apparatus, wherein at least a portion of the storage device includes a protective lining configured to protect the second area of the collection apparatus from one or more environmental factors.

24. A method for manufacturing a system for collecting and storing one or more biometric identifiers, the method comprising:
forming a collection document having a portion configured to be positioned in a mouth of a first user, the collection document having a first panel, a second panel, and a third panel, wherein the portion is located at least in part on the third panel, and wherein the portion is configured to collect a first biometric identifier of the first user while the collection document is positioned within the mouth of the first user;
wherein the collection document is formed such that the collection document may be adjusted from an open configuration into a closed configuration by adjusting the collection document comprising:
folding the third panel such that a first face of the third panel is positionally opposing a first face of the second panel; and
folding the first panel such that a first face of the first panel is positionally opposing a second face of the third panel, wherein the first biometric identifier that is deposited on the third panel is positionally opposing a first section of the first face of the second panel that does not include a biometric identifier such that the third panel is positioned between the first panel and the second panel while in the closed configuration and such that the portion of the third panel with the first biometric identifier of the first user is at least partially protected from one or more environmental factors.

25. The method of claim 24, wherein the one or more environmental factors comprise one or more of light, moisture, heat, humidity, contaminants, and dust.

26. The method of claim 24, wherein the collection document is configured to be adjusted into an open configuration, such that the portion of the third panel is positioned outside of the first panel and the second panel.

27. The method of claim 24, wherein the collection document comprises a second portion configured to collect a second biometric identifier, wherein the second portion is positioned at least partially upon the second panel such that the second biometric identifier is not in contact with the first biometric identifier when the collection document is in the closed configuration.

28. The method of claim 24, wherein the collection document comprises a second portion where a second biometric identifier may be collected, and wherein the second portion is positioned on the third panel such that the third panel is positioned between the first panel and the second panel while in the closed configuration such that the second portion of the third panel is at least partially protected.
